(19) United States
(12) Patent Application Publication
(10) Pub. No.: US 2025/0272606 A1
(43) Pub. Date: Aug. 28, 2025

(54) SYSTEMS AND METHODS FOR DONOR SELECTION FOR SYNTHETIC CONTROL MODELS

(71) Applicant: Spotify AB, Stockholm (SE)

(72) Inventors: Michael O’RIORDAN, London (GB); Ciarán GILLIGAN-LEE, Dublin (IE)

(73) Assignee: Spotify AB, Stockholm (SE)

(21) Appl. No.: 18/589,139

(22) Filed: Feb. 27, 2024

(51) Int. Cl. G06N 20/00

(52) U.S. Cl. CPC G06N 20/00

(57) ABSTRACT

Systems and methods for donor selection for synthetic control models are provided. When selecting donors for synthetic control models, it is important that the selected donors are not impacted by an intervention. To determine whether potential donors are impacted by the intervention, expected post-intervention values for each donor are determined based on data from before the intervention. The expected values are compared against actual values, and training donors are selected based on the comparisons. A synthetic control model can be trained using the selected training donors.

### BACKGROUND

Synthetic control models are often used to estimate treatment effects in settings with observational time-series data. To identify a causal effect of an intervention on a target unit, a synthetic control model is trained using donor units that are similar to the target unit. However, it is important that the donor units are not impacted by spillover effects (i.e., the donor units are not impacted by the intervention). Further it is important to determine how much potential bias may have been introduced to the synthetic control model from spillover effects.

### SUMMARY

In general terms, this disclosure is directed to systems and methods for donor selection for synthetic control models. In example aspects, expected post-intervention values are determined for a set of potential donors based on data from before an intervention. The expected values are compared against actual values, and training donors are selected from the set of potential donors based on the comparisons. A synthetic control model can be trained using the selected training donors.

In a first aspect, a method for selecting training donors for a synthetic control model is provided. A set of potential donors is determined. Each potential donor is associated with timeseries data comprising data before an intervention and data after the intervention. For each potential donor in the set of potential donors, an expected post-intervention value is determined and compared to an actual post-intervention value for the potential donor. A set of training donors is selected from the set of potential donors based on the comparisons. A synthetic control model is trained on the timeseries data associated with each trained donor in the set of training donors. A visual output device of a computing device presents a graphical representation based, at least in part, on an observed outcome and the synthetic control model.

In a second aspect, a system for selecting training donors for a synthetic control model is provided. The system includes one or more processors and one or more computer-readable storage devices storing data instructions. When the data instructions are executed by the one or more processors, the data instructions cause the system to determine a set of potential donors, determine an expected post-intervention value and compare the expected post-intervention value to an actual post-intervention value for each potential donor, select a set of training donors from the set of potential donors, train a synthetic control model, and cause a visual output device of a computing device to present a graphical representation. Each potential donor is associated with timeseries data comprising data before an intervention and data after the intervention. The set of training donors is selected based on the comparisons between the expected post-intervention values and the actual post-interventions values of the potential donors. The synthetic control model is trained on the timeseries data associated with each training donor in the set of training donors. The graphical representation is based, at least in part, on an observed outcome and the synthetic control model.

In a third aspect, a non-transitory computer-readable medium is provided. The computer-readable medium stores data instructions that, when executed by one or more processors, cause the one or more processors to determine a set of potential donors, determine an expected post-intervention value and compare the expected post-intervention value to an actual post-intervention value for each potential donor, select a set of training donors from the set of potential donors, train a synthetic control model, and cause a visual output device of a computing device to present a graphical representation. Each potential donor is associated with timeseries data comprising data before an intervention and data after the intervention. The set of training donors is selected based on the comparisons between the expected post-intervention values and the actual post-interventions values of the potential donors. The synthetic control model is trained on the timeseries data associated with each training donor in the set of training donors. The graphical representation is based, at least in part, on an observed outcome and the synthetic control model.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

FIG. 1 illustrates a schematic block diagram of an example media playback system for selecting donors for synthetic control models.

FIG. 2 illustrates an example timeline of an addition of an additional feature to a module of a media playback application.

FIG. 3 illustrates an example of a directed acyclic graph on which a synthetic control model may be based.

FIG. 4 illustrates an additional example of a directed acyclic graph on which a synthetic control model may be based.

FIG. 5 illustrates a schematic block diagram of another example of a media playback system shown in FIG. 1.

FIG. 6 illustrates a schematic block diagram of an embodiment of a synthetic control engine.

FIG. 7 illustrates an example donor forecast graph.

FIG. 8 illustrates a first example graph including data from a synthetic control model in a first scenario.

FIG. 9 illustrates a table including data from a synthetic control model in a first scenario.

FIG. 10 illustrates a second example graph including data from a synthetic control model in a first scenario.

FIG. 11 illustrates a third example graph including data from a synthetic control model in a first scenario.

FIG. 12 illustrates a fourth example graph including data from a synthetic control model in a first scenario.

FIG. 13 illustrates a first example graph including data from a synthetic control model in a second scenario.

FIG. 14 illustrates a table including data from a synthetic control model in a second scenario.

FIG. 15 illustrates a second example graph including data from a synthetic control model in a second scenario.

FIG. 16 illustrates a third example graph including data from a synthetic control model in a second scenario.

FIG. 17 illustrates a fourth example graph including data from a synthetic control model in a second scenario.

FIG. 18 illustrates a flowchart of an example method for selecting donors for a synthetic control model.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

As used herein, the term “including” as used herein should be read to mean “including, without limitation,” “including but not limited to,” or the like.

As briefly described above, embodiments of the present disclosure are directed to systems and methods for donor selection for synthetic control models. While described in the context of a synthetic control model for use in software experimentation—such as determining an effect of a new feature in a media playback application, for example—the systems and methods described herein are applicable in any context in which a synthetic control model may be used.

In example aspects, donors are selected by comparing expected values for the donors at or after an intervention to the actual values for the donors at or after the intervention. In an example, the expected values are determined based on time-series data associated with the donors from before the intervention. By comparing the expected values to the actual values, potential spillover effects from the intervention on the donors can be detected, and donors that are not impacted by the intervention can be selected.

In further aspects, one or more bias bounds are computed for the synthetic model. In an example, bias bounds are computed that represent the potential bias from false positives—i.e., the bias if all donors acting as proxies for a relevant latent variable were excluded by the selection procedure. In another example, the bias bounds are computed that represent the potential bias from false negatives—i.e., the bias if selected donors are impacted by the intervention. By calculating bias bounds, the validity of a causal effect determined based on the synthetic control model can be verified.

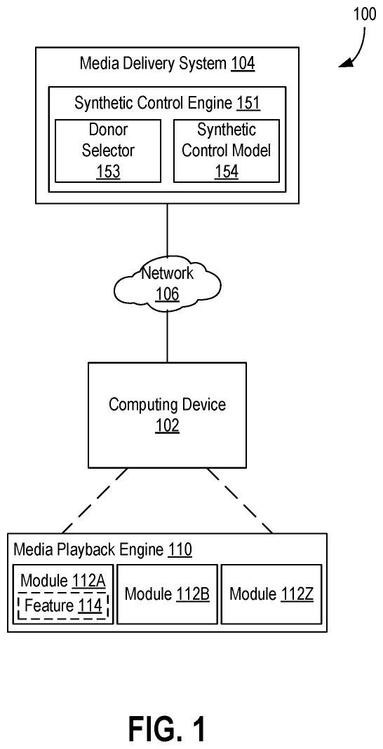

Turning now to FIG. 1, an example media playback system 100 for selecting donors for a synthetic control model is shown. The system 100 includes a media delivery system 104 and a computing device 102. While only one computing device 102 is illustrated, alternative embodiments may include a plurality of computing devices 102. The media delivery system 104 includes a synthetic control engine 151 which includes a donor selector 153 and a synthetic control model 154. The synthetic control model 154 may be used to estimate an effect of, e.g., adding or presenting a new feature 114 within a module 112A of a media playback engine 110. To do this, the synthetic control model 154 is trained using data associated with one or more donors. In an example, the donors are one or more modules 112B, 112Z within the media playback engine 110 that are similar to the module 112A but to which the new feature 114 was not added.

The donor selector 153 selects the donors on which the synthetic control model 154 is trained. In an embodiment, the donor selector 153 selects donors which are not impacted by the intervention, or donors for which the impact of the intervention is minimal. For example, the donor selector 153 may determine expected post-intervention values for a set of potential donors. In an example, the post-intervention values are expected values at a time of an intervention. These expected values may then be compared to actual values at the time of the intervention for the potential donors to determine which of the donors are not impacted by spillover effects (or are minimally impacted by spillover effects). In an alternative example, the post-intervention vales are expected values at a time after the intervention for the set of potential donors, and these expected values can then be compared against corresponding actual values from after the intervention. In some embodiments, the donor selector 153 determines multiple expected values for each potential donor.

The donor selector 153 selects donors by comparing the expected values to the actual values. Because the expected values are determined assuming that the potential donors are not impacted by the intervention, differences between the expected values and the actual values can be assumed to be due to spillover effects. Accordingly, in an example, the donor selector 153 selects the potential donors that have the smallest differences between the expected values and the corresponding actual values. The synthetic control model 154 can then be trained on the selected training donors.

Once the synthetic control model 154 is trained, the synthetic control engine 151 performs sensitivity analysis on the synthetic control model 154 to determine trustworthiness of a causal effect determined using the synthetic control model 154. In embodiments, the synthetic control engine 151 calculated one or more bias bounds with which the validity of the causal effect can be determined. In an embodiment, the synthetic control engine 151 performs sensitivity analysis as described in U.S. patent application Ser. No. 18/427,475, filed on Jan. 30, 2024, the disclosure of which is hereby incorporated by reference in its entirety.

For example, if an average difference between an observed outcome and a synthetic control unit output by the synthetic control model 154 is within the bias bounds, the effect estimated using the synthetic control model may be untrustworthy. This is because if the average difference between the observed outcome and the synthetic control unit is within the bias bounds, it is possible that with the worst possible bias, the average difference between the observed outcome and the synthetic control unit could be either positive or negative and, therefore, the effect of adding the new feature is indeterminate. For example, if the average difference between the observed outcome and the synthetic control unit is 500, but the bias bounds is +1000, then the range for the average difference between the observed outcome and the synthetic control unit is between −500 to 1500. Because both a positive effect and a negative effect are within the range for the average difference between the observed outcome and the synthetic control unit, the effect of adding the new feature is indeterminate. In such a case, the donor selector 153 may select a new set of donors, and the synthetic control model 154 may be retrained on the new set of donors. Alternatively, if the average difference between the observed outcome and the synthetic control unit output by the synthetic control model 154 is outside of the bias bounds, the causal effect estimated using the synthetic control model may be trustworthy.

In some embodiments, multiple bias bounds may be calculated, as described further herein. In such embodiments, the causal effect estimated using the synthetic control model may be trustworthy if the average difference between the observed outcome and the synthetic control unit output by the synthetic control model 154 is outside each of the bias bounds.

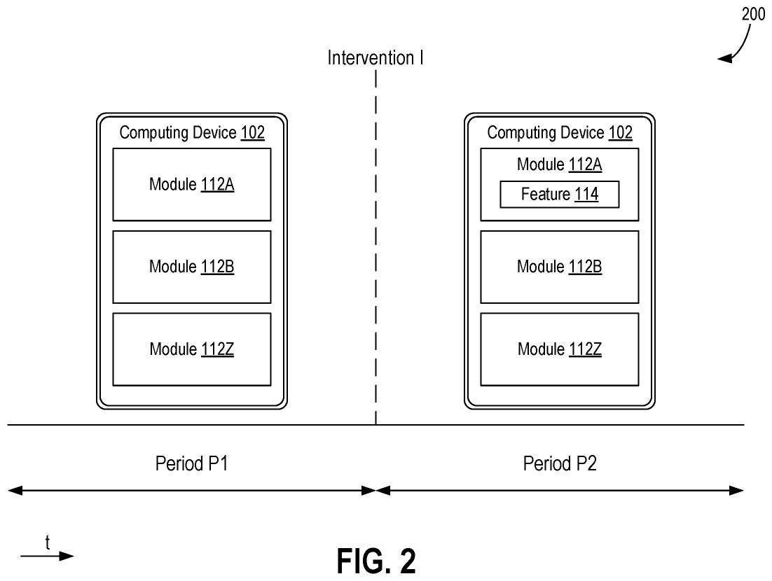

FIG. 2 illustrates a timeline 200 of an addition of a new feature within a media playback application. During a first period P1, the media playback application has a plurality of modules 112. At an intervention I, a new feature 114 is added to the module 112A. Data on the use of the media playback application and the specific modules 112 may be collected during the period P1 and after the intervention I during a period P2. For example, the data collected could be an amount of time that users use the different modules during each of the periods P1, P2.

As explained herein, a donor selector may evaluate the collected data to select donors on which a synthetic control model may be trained. In an example, the modules 112B, 112Z are used as donors to predict a synthetic control unit that represents what would have happened with the module 112A during the period P2 if the new feature 114 had not been added. In the example described above, the synthetic control unit could represent an amount of time that users would have used the module 112A if the new feature 114 had not been added. This synthetic control unit can then be compared to the observed outcome that occurs during the period P2—i.e., the observed amount of time that users used the module 112A—to determine the effect of the intervention I—i.e., the addition of the new feature 114.

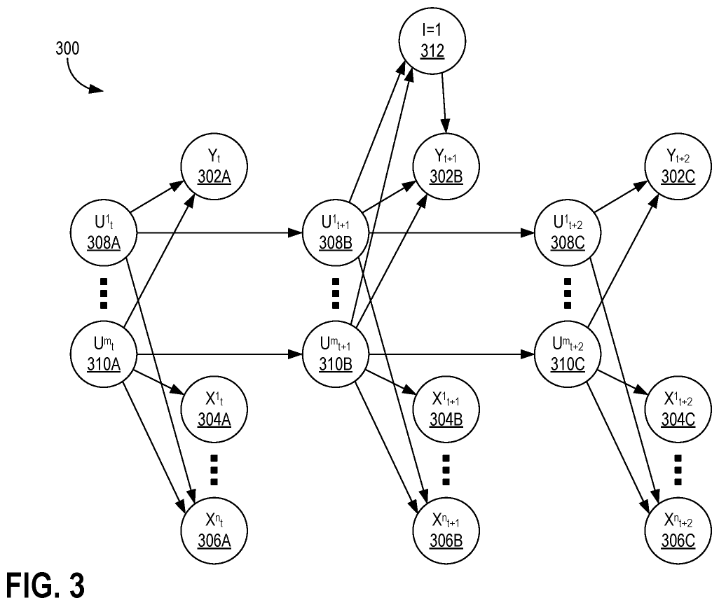

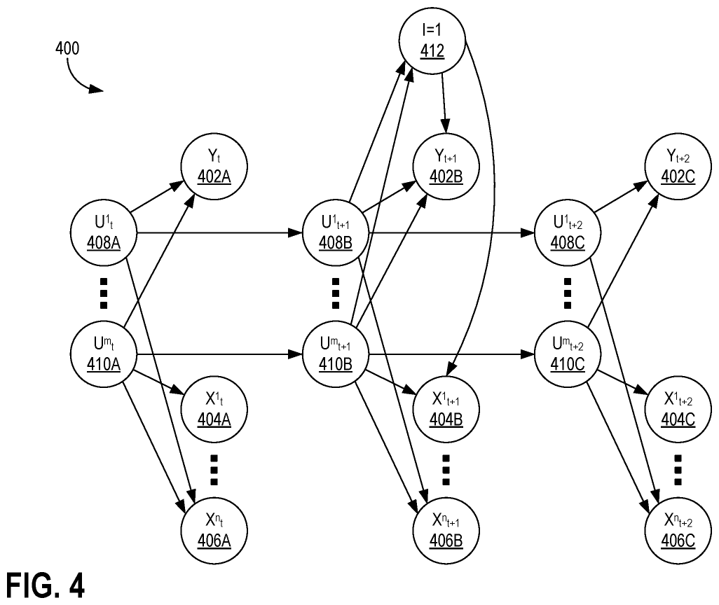

FIGS. 3 and 4 illustrate examples of directed acyclic graphs 300, 400 on which a synthetic control model in a synthetic control engine (such as the synthetic control model 154 in the synthetic control engine 151 of FIG. 1) may be based.

The graph 300 illustrated in FIG. 3 illustrates a directed acyclic graph in which each of the donors are valid donors for a synthetic control model—i.e., the donors are not impacted by the intervention. The graph 300 include a plurality of nodes and arrows, with the arrows representing causal effects caused by nodes on other nodes.

Target nodes 302A-C represent a target at various points in time. For example, the target may be usage of a module that receives an additional feature in a media playback application. A first node 302A from this group may represent usage of the module before the additional feature is added, while other nodes 302B-C may represent usage of the module after the additional feature is added.

Donor nodes 304A-C, 306A-C may represent donors at various points in time. As described above, donors may be similar to the target, but may not be affected by an intervention. For example, the donors may be usage data for other modules in the media playback application that do not receive the additional feature. Like with the nodes 302A-C representing the target, the nodes 304A-C, 306A-C may represent the donors at various points in time, both before and after the intervention.

Latent cause nodes 308A-C, 310A-C may represent latent causes. These latent causes are variables that have causal effects on the target and the donors. For example, these nodes 308A-C, 310A-C may represent reasons that users may wish to use the modules of the media playback application. These latent causes may affect both the target nodes 302A-C and the donor nodes 304A-C, 306A-C.

An intervention node 312 may represent the intervention. For example, this may be the addition of the additional feature to the target module in the media playback application. In the graph 300, the intervention node 312 impacts the target node 302B, but does not impact the donor nodes 304B, 306B. Accordingly, the donor nodes 304A-C, 306A-C are valid donors on which a synthetic control model may be trained.

The graph 400 illustrated in FIG. 4 is substantially similar to the graph 300 illustrated in FIG. 3; however, the graph 400 shows a directed acyclic graph in which one of the donors is not a valid donor for a synthetic control model—i.e., a donor is impacted by the intervention.

In the illustrated example, the intervention 412 impacts both the target node 402B and the donor node 404B. Because the donor node 404B is impacted by spillover effects, the donor nodes 404A-C may not be a valid donor on which a synthetic model may be trained. In some embodiments, if the impact of the intervention node 412 on the donor node 404B is minimal, the donor nodes 404A-C may still be used as a donor on which the synthetic model is trained. Processes are described further herein for determining whether a donor has been impacted by the intervention, and selecting donors based on this determination.

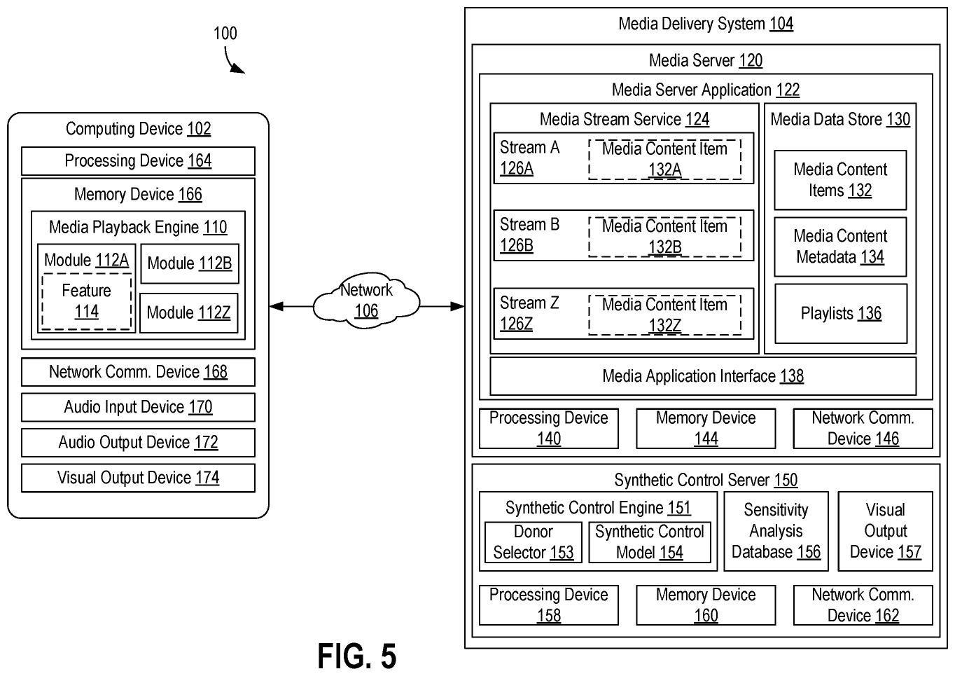

FIG. 5 illustrates a schematic block diagram illustrating another example of the media playback system 100 shown in FIG. 1. In this example, the media playback system 100 includes the computing device 102 and the media delivery system 104. The network 106 is also shown for communication between the computing device 102 and the media delivery system 104.

As described herein, the computing device 102 operates to play media content items using a media playback engine that includes multiple modules 112, which may include features 114. In some embodiments, the computing device 102 operates to play media content items 132 that are provided (e.g., streamed, transmitted, etc.) by a system remote from the computing device 102 such as the media delivery system 104, another system, or a peer device. Alternatively, in some embodiments, the computing device 102 operates to play media content items stored locally on the computing device 102. Further, in at least some embodiments, the computing device 102 operates to play media content items that are stored locally as well as media content items provided by remote systems.

In some embodiments, the computing device 102 includes a processing device 164, a memory device 166, a network communication device 168, an audio input device 170, an audio output device 172, and a visual output device 174. In the illustrated example, the memory device 166 includes the media playback engine 110 and its modules 112 and the modules' features 114. Other embodiments of the computing device 102 include additional, fewer, or different components. Examples of computing devices include a smartphone, a smart speaker, and a computer (e.g., desktop, laptop, tablet, etc.).

In some embodiments, the processing device 164 comprises one or more processing devices, such as central processing units (CPU). In other embodiments, the processing device 164 additionally or alternatively includes one or more digital signal processors, field-programmable gate arrays, or other electronic circuits. In some embodiments, the processing device 164 includes at least one processing device that can execute program instructions to cause the at least one processing device to perform one or more functions, methods, or steps as described herein.

The memory device 166 operates to store data and program instructions. In some embodiments, the memory device 166 stores program instructions for the media playback engine 110 that enables playback of media content items received from the media delivery system 104, and for the modules 112 and their features 114. As described herein, the media playback engine 110 is configured to communicate with the media delivery system 104 to receive one or more media content items—e.g., through the media content streams 126 (including media content streams 126A, 126B, and 126Z).

The memory device 166 includes at least one memory device. The memory device 166 typically includes at least some form of computer-readable media. Computer readable media include any available media that can be accessed by the computing device 102. By way of example, computer-readable media can include computer readable storage media and computer readable communication media.

Computer readable storage media includes volatile and nonvolatile, removable and non-removable media implemented in any device configured to store information such as computer readable instructions, data structures, program modules, or other data. Computer readable storage media includes, but is not limited to, random access memory, read only memory, electrically erasable programmable read only memory, flash memory and other memory technology, compact disc read only memory, blue ray discs, digital versatile discs or other optical storage, magnetic cassettes, magnetic tape, magnetic disk storage or other magnetic storage devices, or any other medium that can be accessed by the computing device 102. In some embodiments, computer readable storage media is non-transitory computer readable storage media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The network communication device 168 is a device that operates to communicate data across the network 106. The network communication device 168 allows the computing device 102 to communication with remote devices, such as with the media server 120 and the synthetic control server 150 of the media delivery system 104. Examples of the network communication device 168 include wired and wireless data communication devices, such as a cellular, WIFI, BLUETOOTH™, LoRa, and wired (e.g., Ethernet) communication device.

Some embodiments include an audio input device 170 that operates to receive audio input, such as voice input provided by the user. The audio input device 170 typically includes at least one microphone. In some embodiments, the audio input device 170 detects audio signals directly, and in other embodiments, the audio input device 170 communicates with another device that detects the audio signals (such as through a Bluetooth-connected microphone).

The audio output device 172 operates to output audible sounds, such as the media content and other audio outputs, such as audio cues. In some embodiments, the audio output device 172 generates media output to play media content. Examples of the audio output device 172 include a speaker, an audio output jack, and a Bluetooth transceiver (such as for communication with a Bluetooth-connected speaker). In some embodiments, the audio output device 172 generates an audio output directly, and in other embodiments, the audio output device 172 communicates with another device that generates the audio output. For example, the audio output device 172 may transmit a signal through an audio output jack or a Bluetooth transmitter that can be used to generate the audio signal by a connected or paired device such as headphones or a speaker.

Some embodiments also include a visual output device 174. The visual output device 174 includes one or more light-emitting devices that generate a visual output. Examples of the visual output device 174 includes a display device (which can include a tough-sensitive display device) and lights such as one-or-more light-emitting diodes (LEDs).

Still with reference to FIG. 5, the media delivery system 104 includes one or more computing devices, such as the media server 120 that provides media content items 132 to the computing device 102, and the synthetic control server 150 that selects donors for a synthetic control model 154, train the synthetic control model 154, and performs sensitivity analysis on the synthetic control model 154. Each of the media server 120 and the synthetic control server 150 can include multiple computing devices in some embodiments. Although shown as separate servers, the media server 120 and the synthetic control server 150 may be the same server.

In some embodiments, the media delivery system 104 operates to transmit media content items 132 to one or more media playback devices such as the computing device 102.

In this example, the media server 120 comprises a media server application 122, a processing device 140, a memory device 144, and a network communication device 146. The processing device 140, memory device 144, and network communication device 146 may be similar to the processing device 164, memory device 166, and network communication device 168 respectively, which have been previously described.

In some embodiments, the media server application 122 operates to stream music or other audio, video, or other forms of media content. The media server application 122 includes a media stream service 124, a media data store 130, and a media application interface 138.

The media stream service 124 operates to buffer media content such as media content items 132 (including 132A, 132B, and 132Z) for streaming to one or more streams 126 (including 126A, 126B, and 126Z).

The media application interface 138 can receive requests or other communication from the media playback devices (such as the computing device 102) or other systems, to retrieve media content items from the media delivery system 104. For example, in FIG. 5, the media application interface 138 receives communications from the media playback engine 110 of the computing device 102.

In some embodiments, the media data store 130 stores media content items 132, media content metadata 134, and playlists 136. The media data store 130 may comprise one or more databases and file systems. Other embodiments are possible as well. As noted above, the media content items 132 may be audio, video, or any other type of media content, which may be stored in any format for storing media content. For example, media content items 132 may be songs, audiobooks, podcasts, or advertisements.

The media content metadata 134 operates to provide information associated with the media content items 132. In some embodiments, the media content metadata 134 includes one or more of title, artist, lyrics, album name, length, genre, mood, era, or other media metadata, as described herein.

The playlists 136 operate to identify one or more of the media content items 132. In some embodiments, the playlists 136 identify a group of the media content items 132 in a particular order. In other embodiments, the playlists 136 merely identify a group of the media content items 132 without specifying a particular order. Some, but not necessarily all, of the media content items 132 included in a particular one of the playlist 136 are associated with a common characteristic such as a common genre, mood, or era.

In this example, the synthetic control server 150 includes a synthetic control engine 151 that has a donor selector 153 and a synthetic control model 154, a synthetic control database 156, a visual output device 157, a processing device 158, a memory device 160, and a network communication device 162.

In some embodiments, any one or more of the functions, methods, and operations described herein as being performed by the synthetic control server 150—or components of the synthetic control server 150, such as the synthetic control engine 151—can alternatively be performed by the computing device 102. This may include embodiments where the media delivery system 104 does not include a synthetic control server 150 and embodiments where the synthetic control server 150 cooperates with the computing device 102 and the functions are split between those components.

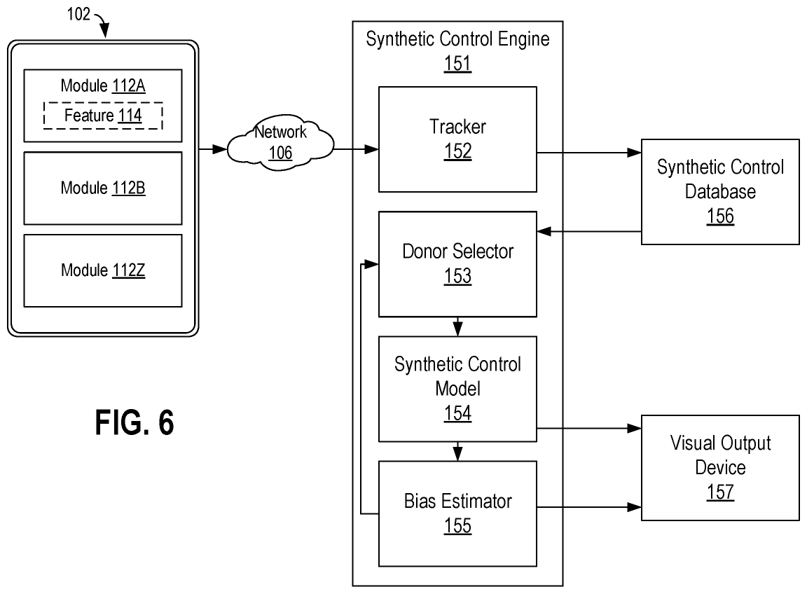

The synthetic control engine 151 can operate on a single computing device, or by cooperation of multiple computing devices. For example, the synthetic control engine 151 can operate solely on the computing device 102 or solely on the synthetic control server 150. Alternatively, portions of the synthetic control engine 151 can be performed by one or more other computing devices, such as by data communication between the computing device 102 and the media delivery system 104. In the example shown in FIG. 5, the media delivery system 104 includes the synthetic control engine 151. The synthetic control engine 151 can perform any one or more of the operations described herein, such as with reference to FIG. 6.

The processing device 158, memory device 160, and network communication device 162 may be similar to the processing device 164, memory device 166, and network communication device 168 respectively, which have each been previously described.

In various embodiments, the network 106 includes one or more data communication links, which may include multiple different types. For example, the network 106, can include wired and/or wireless links, including BLUETOOTH™, ultra-wideband (UWB), 802.11, ZigBee, cellular, LoRa, and other types of wireless links. Furthermore, in various embodiments, the network 106 is implemented at various scales. For example, the network 106 can be implemented as one or more local area networks (LANs), metropolitan area networks, subnets, wide area networks (such as the Internet), or can be implemented at another scale. Further, in some embodiments, the network 106 includes multiple networks, which may be of the same type or of multiple different types.

Although FIG. 5 illustrates only a single computing device 102 in communication with a single media delivery system 104, in accordance with some embodiments, the media delivery system 104 can support the simultaneous use of multiple computing devices 102. Additionally, the computing device 102 can simultaneously access media content from multiple media delivery systems 104.

FIG. 6 illustrates an example embodiment of a synthetic control engine 151. In the illustrated embodiment, the synthetic control engine 151 includes a tracker 152, a donor selector 153, a synthetic control model 154, and a bias estimator 155. The synthetic control engine 151 may also be connected with a synthetic control database 156 and a visual output device 157. The synthetic control engine 151 may also communicate with a computing device 102 over a network 106. The computing device 102 may have a plurality of modules 112, which may include a feature 114. Although not depicted in FIG. 6, the modules 112 may be part of a media playback engine.

The synthetic control engine 151 may operate to collect data from the computing device 102 for training the synthetic control model 154. This data may be time-series data including data from a first period before an intervention and from a second period after the intervention. For example, the tracker 153 may collect time-series data from the computing device 102 relating to the usage of the modules 112, such as a number of minutes each module 112 is used. Like as is shown in FIG. 2, a feature 114 may be added to one of the modules 112A on the computing device 102. The tracker 153 may collect time-series data relating to the usage of the modules 112 both before the feature 114 is added and after the feature 114 is added. This data may be stored by the tracker 153 in the synthetic control database 156.

To train the synthetic control model 154, donors are selected. The donors may be similar to the target but may not have been affected by the intervention. For example, the donors may be modules 112 that are similar to the module 112A but did not have the feature 114 added to them. In the illustrated embodiment, the module 112B and the module 112Z may be selected as donors on which the synthetic control model 154 may be trained.

The donor selector 153 may evaluate the collected data to select donors on which the synthetic control model 154 can be trained. The donor selector 153 may select donors that are valid—i.e., donors that are not impacted by spillover effects from the intervention. In an example, spillover effects are manifested as post intervention shifts in donor errors. Accordingly, the donor selector 153 may detect time-series data from before the intervention to test for shifts in the donor errors that would rule out donors as valid donors.

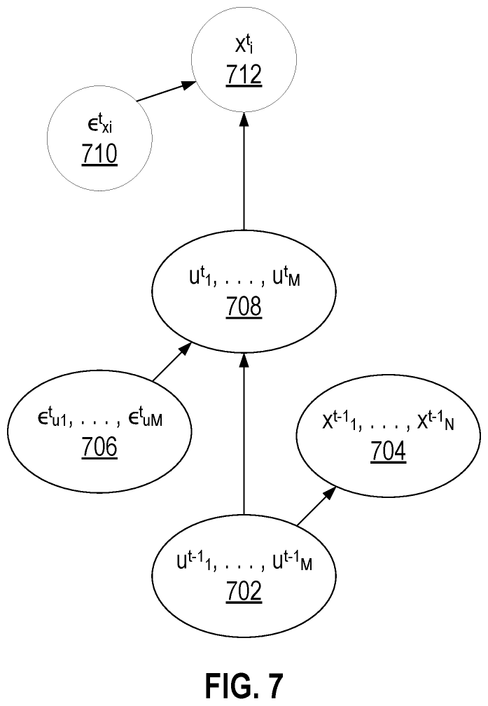

FIG. 7 illustrates an example donor forecast 700. The illustrated donor forecast 700 shows donors x₁^{t-1} through x_{N}^{t-1} 704 as proxies for latent variables u₁^{t-1} through u_{M}^{t-1} 702 at time t−1. Based on this donor forecast 700, at time t, each donor xᵢ^{t} 712 can be represented as a function hᵢ of the donors at time t−1 and noise terms ϵₓᵢ^{t} 710 (e.g., error distributions for the donors) and ϵᵤ^{t} 706 (e.g., error distributions for the latent variables) according to the following equation:

$$1 ) 𝔼 ( {x}_{i}^{t} ) = 𝔼 ( {h}_{i} ( {x}_{1}^{t - 1} , … , {x}_{N}^{t - 1} , P ( {ϵ}_{{x}_{i}}^{t} , {ϵ}_{u}^{t} ) ) )$$

Using this equation, post-intervention donor values can be estimated based on pre-intervention donor data. In an example, an estimated value for a donor can be determined at the time of intervention t. Using pre-intervention data at time points 1<t′<t, the function hᵢ can be learned by minimizing the following function:

$$2 ) 𝔼 ( {x}_{1}^{{t}^{′}} - {h}_{i} ( {x}_{1}^{{t}^{′} - 1} , … , {x}_{N}^{{t}^{′} - 1} ) )$$

The learned function ĥᵢ can be used to predict the estimated value for each donor \hat{x}ᵢ using the following equation:

$$3 ) {\hat{x}}_{i}^{t} = {\hat{h}}_{i} ( {x}_{1}^{t - 1} , … , {x}_{N}^{t - 1} )$$

Returning to FIG. 6, the donor selector 153 selects donors using the processes described above. In an example, the synthetic control model 154 is a linear synthetic control model. In this example, the following linear model for forecasting future values for a donor xᵢ may be used by the donor selector 153 and the synthetic control model 154:

$$4 ) {x}_{i}^{{t}^{′}} ∼ 𝒩 ( {α}_{i} + {\sum }_{j} {β}_{iN} {x}_{N}^{{t}^{′} - 1} , {σ}_{{x}_{i}} )$$

Based on this model, the donor selector 153 may select donors on which the synthetic control model 154 can be trained. In an embodiment, the donor selector 153 selects a predetermined number of donors based with the smallest difference between the estimated values and the actual values. For example, the donor selector 153 may rank the donors based on the following equation and select a predetermined number of donors based on the ranking:

$$5 ) {min}_{{x}_{i}} ( \vert {x}_{i}^{t} - {\sum }_{j} {β}_{iN} {x}_{N}^{t - 1} \vert )$$

In an alternative embodiment, the donor selector 153 selects donors for which a difference between the expected value and the actual value is less than a threshold. For example, the donor selector 153 selects donors for which the actual values fall within predetermined posterior predictive intervals for the estimated values (e.g., 80% posterior predictive intervals). In an embodiment, each donor for which the difference between the expected value and the actual value is less than the threshold is selected. Alternatively, a subset of the donors for which the difference between the expected value and the actual value is less than the threshold may be selected.

While this example describes using a linear synthetic control model, in alternative examples, any flexible machine learning model can be used. In an example, the linear model can be swapped for the flexible machine learning model using conformal inference for constructing calibrated prediction intervals.

Using the data collected on the usage of the modules 112, the synthetic control model 154 may be trained to determine an effect on a target. The synthetic control model 154 may generate a synthetic control unit that simulates what would have happened with the target had the intervention not occurred. For example, the synthetic control model 154 may be trained to determine an effect of adding the feature 114 to the module 112A, and the synthetic control unit may simulate usage of the module 112A if the feature 114 had not been added to it. The synthetic control model 154 may be trained using the synthetic control engine 151.

The synthetic control model 154 may be trained using time-series data associated with the selected donors in any way currently known in the art. For example, training the synthetic control model may include determining weights for calculating a weighted average of the donor time-series data that mirrors the target time-series data as close as possible during the first period before the intervention. This may be done, for example, using linear regression without intercept and allowing for negative coefficients.

Once the synthetic control model 154 has been trained, the bias estimator 155 may calculate one or more bias bounds for the synthetic control model 154. In an embodiment, first bias bounds may be calculated using data from the synthetic control model 154 based on an assumption that the synthetic control model 154 includes the most important latent cause, and any latent cause not considered when training the synthetic control model 154 will at most be as impactful as the most important latent cause. In an embodiment, the first bias bounds are calculated as a product of a number of donors in the synthetic control model 154, a maximum weight in the synthetic control model 154, and a maximum of the average differences in the data from before the intervention and after the intervention (e.g., before the feature 114 was added and after the feature 114 was added to the module 112A) for each donor. In an embodiment, the number of donors used does not include donors in which a weight associated with the donor is zero. Similarly, the average difference in data from before the intervention and after the intervention may not be considered for donors associated with a weight that is zero.

For example, the module 112B and the module 112Z may be selected as donors. After the synthetic control model 154 is trained using the module 112B and the module 112Z, the synthetic control model 154 may include two learned weights with the following values: 2 and 6. Additionally, in a period before the feature 114 was added to the module 112A, the module 112B had an average of 10 minutes of usage and the module 112Z had an average of 8 minutes of usage, and in a period after the feature 114 was added to the module 112A, the module 112B had an average of 11 minutes of usage and the module 112Z had an average of 6 minutes of usage. In this example, the number of donors is 2, the maximum weight in the synthetic control model 154 is 6, and the maximum of the average differences in the data from before the feature 114 was added and after the feature 114 was added to the module 112A for each donor is 2 (the average difference for the module 112Z being the difference between 8 and 6). Therefore, in this example the first bias bounds would be ±24 minutes.

In embodiments, a second bias bounds may be calculated using data from the synthetic control model 154 to determine potential bias from false positives during the donor selection process—i.e., if donors acting as proxies for a relevant latent variable were excluded by the selection procedure. In an example, because the excluded donors are observed, the assumptions used in the calculation for the second bias bounds may be less than the assumptions used to calculate the first bias bounds described above. In an example, the second bias bounds are calculated as a product of a number of donors in the synthetic control model 154, a maximum weight in the synthetic control model 154, and a maximum of the average differences in the data from before the intervention and after the intervention (e.g., before the feature 114 was added and after the feature 114 was added to the module 112A) for each excluded donor.

For example, the module 112B and the module 112Z may be selected as donors, and a third module and a fourth module may be excluded. Using the same example as above, the number of donors is 2 and the maximum weight in the synthetic control model is 6. In a period before the feature 114 was added to the module 112A, the third module had an average of 14 minutes of usage and the fourth module had an average of 6 minutes of usage, and in a period after the feature 114 was added to the module 112A, the third module had an average of 11 minutes of usage and the fourth module had an average of 7 minutes of usage. In this example, with 2 donors, a maximum weight of 6, and a maximum of the average differences in the data from before the feature 114 was added and after the feature 114 was added to the module 112A for each excluded donor of 3 (the average difference for the third module is the difference between 14 and 11), the second bias bounds would be ±36 minutes.

In embodiments, a third bias bounds may be calculated using data from the synthetic control model 154 to determine potential bias from false negatives during the donor selection process—i.e., if selected donors are impacted by the intervention. In an example, the second bias bounds are calculated as a product of a number of donors in the synthetic control model 154, a maximum weight in the synthetic control model 154, and a maximum spillover effects from the intervention on the selected donors. In an embodiment, the spillover effects for each donor are parameters that may be set by a user, for example by using domain knowledge. In an example, the user may set the spillover effect parameters to judge how large of a spillover effect would be needed in order for the estimated causal effect from the synthetic control model to change sign.

For example, the module 112B and the module 112Z may be selected as donors. Using the same example as above, the number of donors is 2 and the maximum weight in the synthetic control model is 6. In this example the module 112B has a spillover effect of 4 minutes and the module 112Z has a spillover effect of 3 minutes. Accordingly, the third bias bounds would be ±48 minutes.

The bias bounds may be used to determine if the effect of adding the feature 114 to the module 112A determined by the synthetic control model 154 is trustworthy. If an average difference between an observed outcome and the synthetic control unit is outside of the bias bounds, the effect determined using the synthetic control model 154 may be trustworthy. Because the average difference between the observed outcome and the synthetic control unit is outside of the bias bounds, then at every possible bias within the bias bounds, the average difference will always have the same sign. Therefore, even with a worst-case bias, the effect will still be in the same direction.

For example, if the average difference between the observed outcome and the synthetic control unit is 500 and the bias bounds are ±250, then even with a maximum amount of bias, the average difference between the observed outcome and the synthetic control unit may be between 250 and 750. In this case, even with the maximum amount of bias, the average difference between the observed outcome and the synthetic control unit is positive, so a trustworthy positive causal effect may be determined from the synthetic control model 154.

In another example, if the average difference between the observed outcome and the synthetic control unit is 500, but the bias bounds is ±1000, then the range for the average difference between the observed outcome and the synthetic control unit may be between −500 and 1500. Because both a positive effect and a negative effect are within the range for the average difference between the observed outcome and the synthetic control unit, the effect of adding the new feature is indeterminate even though the average difference between the observed outcome and the synthetic control unit would indicate that the effect is positive.

In these examples, a single bias bounds are used. However, the trustworthiness of the causal effect determined using the synthetic control model 154 may be determined using multiple bias bounds. In an example, for the causal effect to be trustworthy, the average difference between the observed outcome and the synthetic control unit must be outside of each of the bias bounds. In embodiments, the multiple bias bounds are compared to each other to determine the largest bias bounds, and the average difference between the observed outcome and the synthetic control unit is compared to the largest bias bounds. If the average difference between the observed outcome and the synthetic control unit is outside of the largest bias bounds, the average difference between the observed outcome and the synthetic control unit is also outside of any other calculated bias bounds. In alternative embodiments, the average difference between the observed outcome and the synthetic control unit is compared against multiple calculated bias bounds.

The bias bounds may also be used to determine if the synthetic control model 154 should be retrained using additional or alternative donors. In an example, if the effect determined from the synthetic control model 154 is indeterminate and not trustworthy, it may be because a latent cause affects the target but does not affect any of the donors on which the synthetic control model 154 was trained, potentially because the donors that act as proxies for the latent cause were excluded by the donor selection process. In such a case, any causal effect determined using the synthetic control model 154 may be due to a shift in this unconsidered latent cause instead of the intervention (e.g., the addition of the new feature 114). In another example, the effect determined using the synthetic control model 154 may be untrustworthy because donors selected during the donor selection process are impacted by spillover effects. To overcome this, new donors may be selected on which the synthetic control model 154 may be trained, and at least one of these new donors may be affected by the previously unconsidered latent cause. In an example, the donor selector 153 may reselect the new donors using the process described above, but the donor selector 153 may select a different number of donors or may use a different threshold to select the new donors.

Output from the synthetic control model 154 and/or from the bias estimator 155 may be shown on the visual output device 158. Data shown on the visual output device 158 may assist a user in determining that the effect determined from the synthetic control model 154 is trustworthy. In embodiments in which the average difference between the observed outcome and the synthetic control unit are within the one or more bias bounds, the data displayed on the visual output device 158 may assist the user in determining that new donors should be selected to retrain the synthetic control model 154.

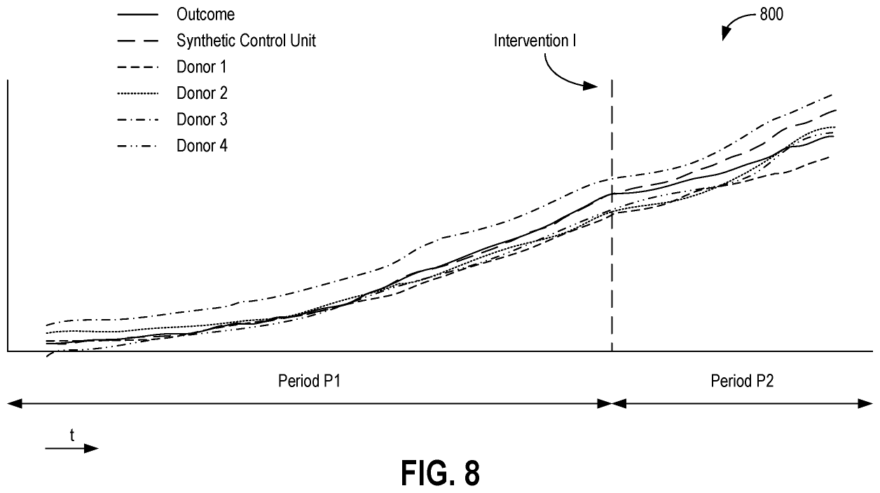

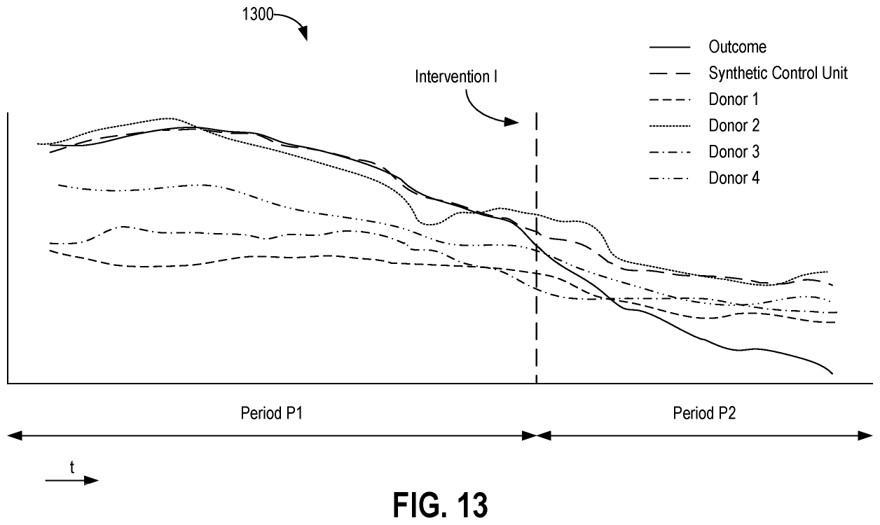

FIGS. 8-17 illustrate examples of data from a synthetic control model and bias estimator displayed on a visual output device. FIGS. 8-12 illustrate data from a first example scenario in which the effect determined using the synthetic control model is not trustworthy, and FIGS. 13-17 illustrate data from a second example scenario in which the effect determined using the synthetic control model is trustworthy.

FIG. 8 illustrates a graph 800 of time-series data that includes data on an observed outcome, a synthetic control unit, and a plurality of donors. The synthetic control unit may be determined using a synthetic control model (such as the synthetic control model 154 described with relation to FIG. 5). The illustrated graph 800 includes time-series data from a first period P1 and a second period P2. An intervention I separate the first period P1 and the second period P2. For example, the intervention I could be an addition of an additional feature to a module in a media playback application. This graph 800 illustrates a difference between the synthetic control unit and the observed outcome, particularly during the second period P2 after the intervention I. The graph 800 also illustrates data for the plurality of donors, providing visual insight into how the synthetic control unit may have been determined. In alternative embodiments, the graph 800 may include data on proxies that were not included as donors to train the synthetic control model.

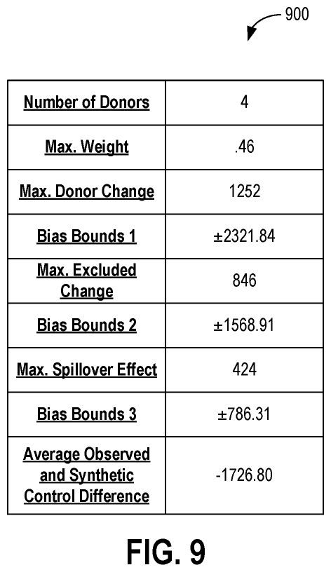

FIG. 9 illustrates a table 900 of data that includes a number of donors, a maximum weight value, a maximum donor change value, first bias bounds, a maximum excluded donor change value, a second bias bounds, a maximum spillover effect, a third bias bounds and an average difference between the observed outcome and the synthetic control unit. As described above, the maximum weight value is a maximum value of weights of a synthetic control model, the maximum donor change is a maximum value of an average difference between pre-intervention and post-intervention data for each donor used in the synthetic control model, the maximum excluded change is a maximum value of an average difference between pre-intervention and post-intervention data for each excluded donor, and the maximum spillover effect is a parameter set by a user. The first bias bounds may be a product of the number of donors, the maximum weight, and the maximum donor change. The second bias bounds may be a product of the number of donors, the maximum weight, and the maximum excluded change. The third bias bounds may be a product of the number of donors, the maximum weight, and the maximum spillover effect.

The data displayed in the table 900 may be used to determine whether a causal effect determined using the synthetic control model is trustworthy. In an example, if the average difference between the observed outcome and the synthetic control unit is inside any of the calculated bias bounds, the causal effect may not be trustworthy. In the illustrated example, the average difference between the observed outcome and the synthetic control unit is within the first bias bounds, so the causal effect may not be trustworthy.

In alternative embodiments, additional or alternative data may be shown in the table 900. In further embodiments, only of subset of the data is shown. For example, the table 900 may include the largest of the first bias bounds, the second bias bounds, and the third bias bounds and the average difference between the observed outcome and the synthetic control unit.

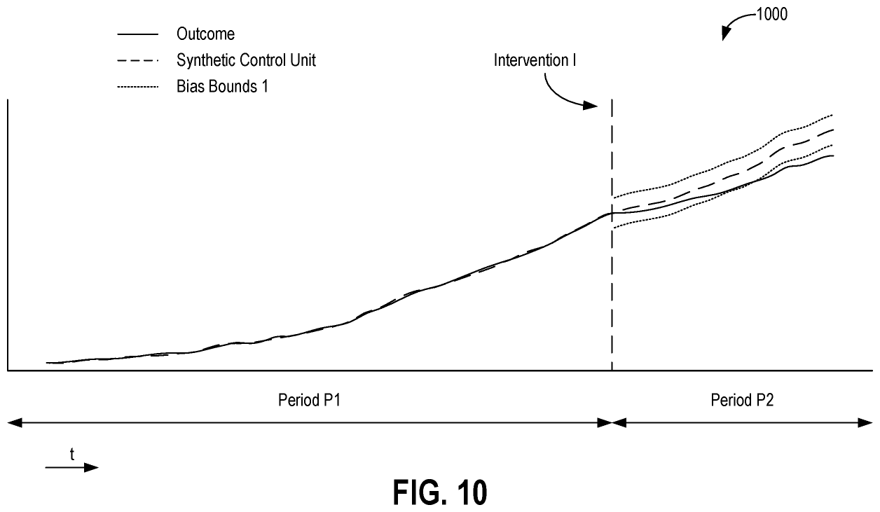

FIG. 10 illustrates a graph 1000 of time-series data that includes data on an observed outcome, a synthetic control unit, and first bias bounds. Like with the graph depicted in FIG. 8, this graph may include data from a first period P1 and a second period P2, the two periods P1, P2 separated by an intervention I. In the illustrated example, the graph 1000 includes only the first bias bounds and not the second bias bounds or the third bias bounds. In embodiments, the graph 1000 includes one calculated bias bounds, such as the largest of the calculated bias bounds. In alternative embodiments, the graph 1000 includes multiple calculated bias bounds. In an example, a user may select which bias bounds to display in the graph 1000. Additionally, in another example, multiple graphs may be shown, each including one of the calculated bias bounds.

By visualizing the synthetic control unit along with bias bounds, a determination may be made as to whether a causal effect determined using the synthetic control model is trustworthy. This may be done, for example, by determining whether more of the synthetic control unit is within the bias bounds or outside of the bias bounds during the period P2. If the synthetic control unit is within the bias bounds more than it is outside of the bias bounds during the period P2, then the effect determined using synthetic control model may be untrustworthy. This is because while the synthetic control unit is within the bias bounds, any difference between the synthetic control unit and the observed outcome could be due to bias and not due to a causal effect of the intervention I. In the illustrated example in FIG. 10, the synthetic control unit is within the bias bounds more than it is outside of the bias bounds. Therefore, it may be determined that the effect determined using synthetic control model is untrustworthy. The same evaluation may be performed in examples in which the graph 1000 includes multiple bias bounds.

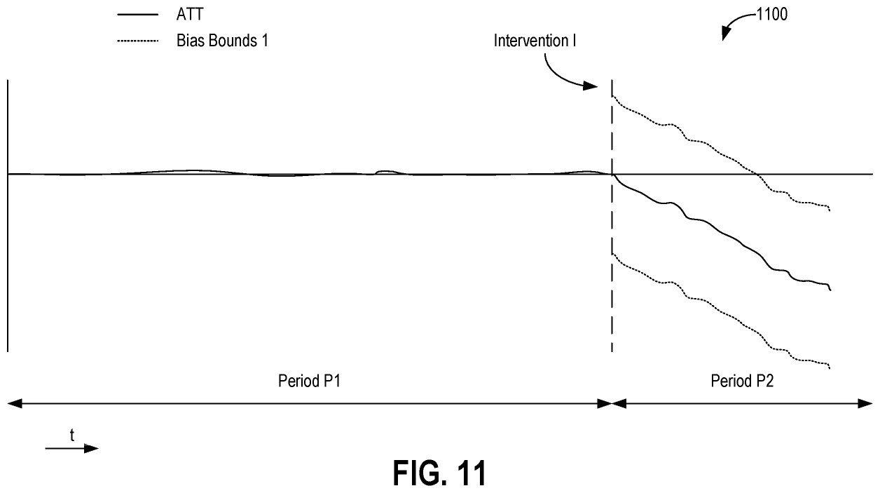

FIG. 11 illustrates a graph 1100 of time-series data that includes an average treatment effect on treated (ATT) and bias bounds. The ATT may be a difference between an observed outcome and a synthetic control unit. As with the previous example graphs, this graph 1100 may include data from a first period P1 and a second period P2, the two periods P1, P2 separated by an intervention I. Additionally, as described above, in alternative embodiments, multiple bias bounds may be displayed in the graph 1100, or multiple graphs may be displayed-a graph for each bias bounds.

This graph 1100 may also be used in making a determination of whether a causal effect determined using the synthetic control unit is trustworthy. This may be done, for example, by determining how long all of the ATT and an upper limit and a lower limit of the bias bounds have the same sign during the period P2. If one of the upper limit or the lower limit of the bias bounds has a different sign than the ATT more than it has the same sign as the ATT during the period P2, then the causal effect determined using the synthetic control unit may be untrustworthy. The same evaluation may be performed in examples in which the graph 1100 includes multiple bias bounds.

In the illustrated example, the ATT is negative for the majority of the period P2, which may indicate a negative causal effect of the intervention I. However, an upper limit of the bias bounds is positive for a majority of the period P2, which may indicate that the negative causal effect is untrustworthy. This is because with the worst possible bias, the causal effect may actually be positive more than it is negative during the period P2.

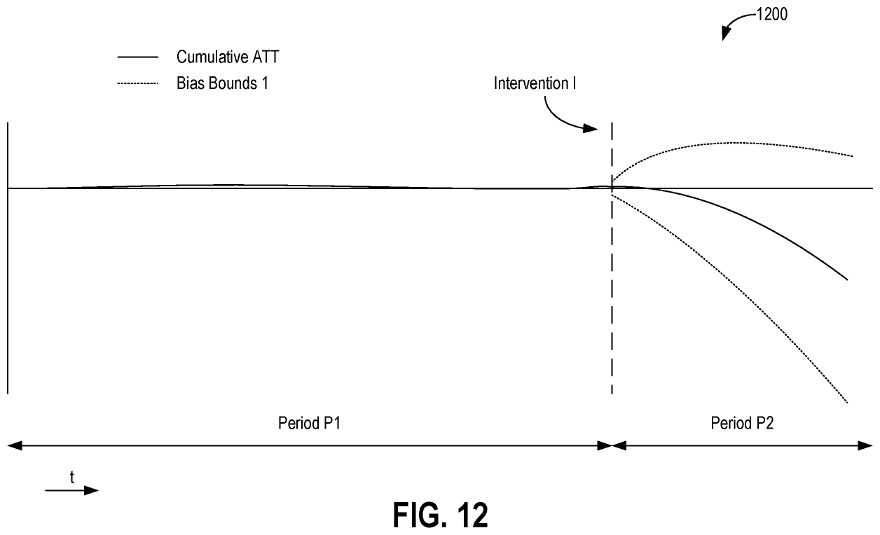

FIG. 12 illustrates a graph 1200 of time-series data that includes a cumulative ATT and bias bounds. The cumulative ATT may represent a sum of all previous values of the ATT. As with the previous example graphs, this graph 1200 may include data from a first period P1 and a second period P2, the two periods P1, P2 separated by an intervention I. Additionally, as described above, in alternative embodiments, multiple bias bounds may be displayed in the graph 1200, or multiple graphs may be displayed, each graph including bias bounds.

As with the other graphs, this graph 1200 may be used to determine if a causal effect using a synthetic control model is trustworthy. This may be done by, for example, determining if the cumulative ATT and both an upper limit and a lower limit of the bias bounds all have the same sign at the end of the period P2. If one of the upper limit or the lower limit has a different sign than the cumulative ATT at the end of the period P2, then the causal effect of the intervention I determined using the synthetic control model may be untrustworthy. The same evaluation may be performed in examples in which the graph 1200 includes multiple bias bounds.

In the illustrated example, the cumulative ATT is negative at the end of the period P2, which may indicate a negative causal effect. However, the upper limit of the bias bounds is still positive at the end of the period P2. Therefore, the negative causal effect of the intervention I determined using the synthetic control model may be untrustworthy. This is because with the worst possible bias, the cumulative ATT may be positive rather than negative, so a determination that the causal effect of the intervention I may be untrustworthy.

While FIGS. 8-12 each illustrate only a single graph or table, in some embodiments, multiple graphs and/or tables may be presented on display (such as visual output device 158 of FIG. 4) for a user.

Turning to FIGS. 13-17, a second set of example graphs 1300, 1500, 1600, 1700 and tables 1400 are shown. In this example, a causal effect determined using a synthetic control model may be trustworthy. In the example graphs 1500, 1600, 1700 each of the calculated bias bounds are displayed.

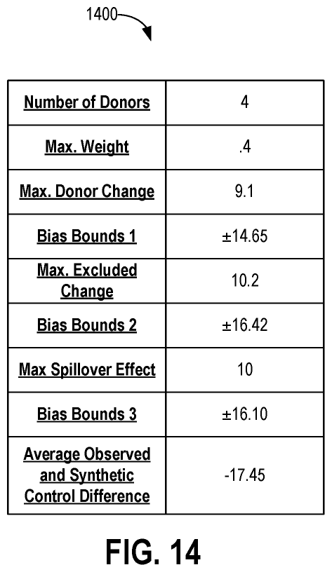

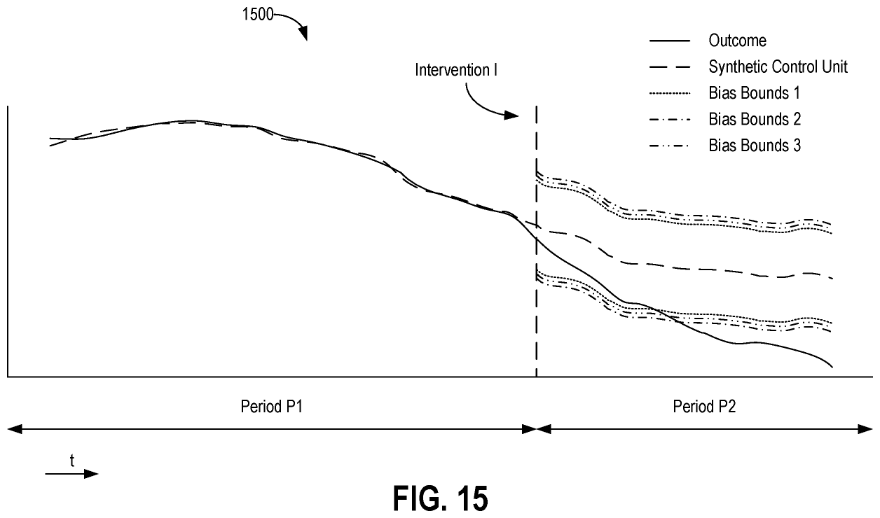

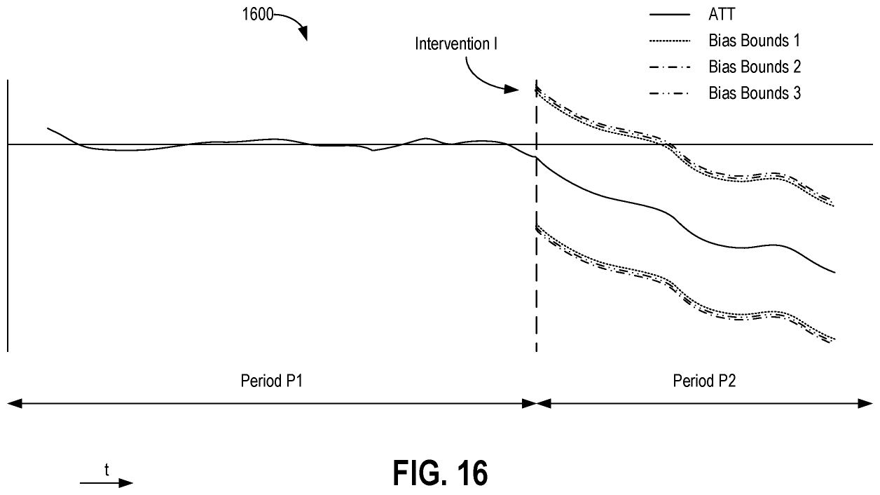

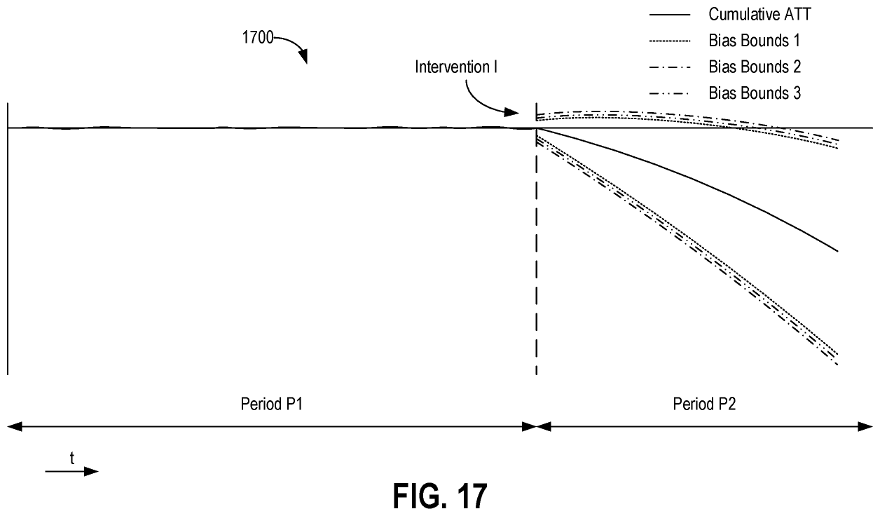

In FIG. 14, the average difference between the observed outcome and the synthetic control unit is outside each of first bias bounds, second bias bounds, and third bias bounds. In FIG. 15, a synthetic control unit is shown to be outside of bias bounds more than it is inside each of the first bias bounds, the second bias bounds, and the third bias bounds during a second period P2 after an intervention I, indicating that the causal effect may be trustworthy. In FIG. 16, all of an ATT and upper limits and lower limits of each of the bias bounds are negative for a majority of the period P2, which may also indicate that the causal effect is trustworthy. In FIG. 17, all of a cumulative ATT and the upper and lower limits of each of the bias bounds are negative at the end of the period P2, which may further indicate that the causal effect may be trustworthy.

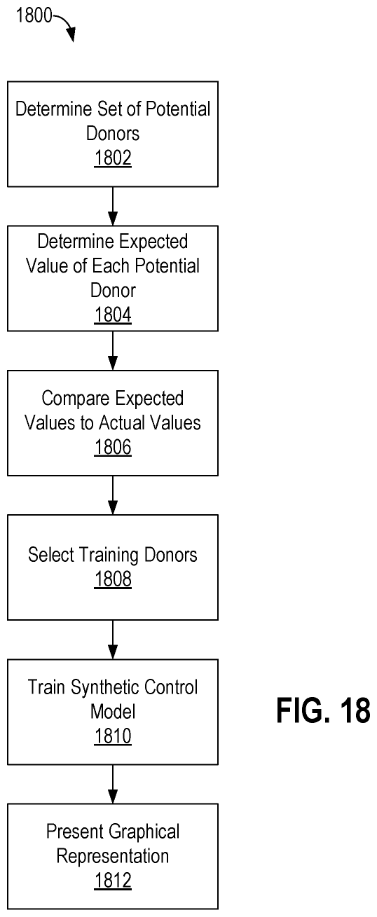

Referring now to FIG. 18, a flowchart of a method 1800 for selecting donors for a synthetic control model is shown. As explained above, the method 1800 may be used, for example, to select donors for a synthetic control model trained to estimate a causal effect of adding a feature in a media playback application. In the illustrated embodiment, the method 1800 includes operations 1802, 1804, 1806, 1808, 1810, and 1812.

The operation 1802 includes determining a set of potential donors. As described above, the set of potential donors may include donors that are similar to a target. In an example, the target is a module to which a feature is added, and the set of potential donors includes other modules to which the feature was not added. In an embodiment, a user selects the set of potential donors. In an alternative embodiment, a donor selector 153 selects the set of donors automatically. For example, the donor selector may evaluate data on a plurality of modules to determine which of the modules are similar to the target module and to which the feature was not added.

The operation 1804 includes determining an expected value for each potential donor. In embodiments, the expected value is a post-intervention value for the donor intervention. For example, the expected value may be a value at a time of an intervention (i.e., a time at which the feature was added) or a value at a time after the intervention. In an alternative example, the expected value is an expected value for the donor after the intervention. In some embodiments, multiple expected values may be calculated for each donor. As described above, time-series data from before the intervention may be used to calculate the expected value. In an embodiment, a donor selector uses data stored in a synthetic control database to determine the expected values of each potential donor.

The operation 1806 includes comparing the expected values determined during the operation 1804 to actual values for each potential donor. In an embodiment, differences between the expected values and the actual values are calculated. In embodiments in which multiple expected values are calculated for each potential donor, each of the multiple expected values are compared against corresponding actual values, and an average may be taken of the differences between the expected values and the actual values. In an embodiment, a donor selector compares the expected values to the actual values.

The operation 1808 includes selecting training donors. In an example, the training donors are selected based on the comparisons in operation 1806. For example, because the expected values are determined assuming that the potential donors are not impacted by the intervention, differences between the expected values and the actual values can be assumed to be due to spillover effects. Accordingly, in this example, potential donors with the smallest differences between the expected values and the actual values may be selected.

In an embodiment, the potential donors are ranked based on the differences between the expected values and the actual values, with the potential donors with smaller differences being ranked higher. A predetermined number of training donors may be selected based on the ranking.

In an alternative embodiment, the differences between the expected values and the actual values are compared to a threshold, and potential donors with a difference lower than the threshold may be selected. In an example, the training donors include potential donors for which the actual values fall within predetermined posterior predictive intervals for the estimated values (e.g., 80% posterior predictive intervals). In embodiments, each of the potential donors which satisfy the threshold are selected. In alternative embodiments, a predetermined number of potential donors which satisfy the threshold are selected.

In an embodiment, a donor selector selects the training donors from the set of potential donors.

The operation 1810 includes training a synthetic control model. The synthetic control model may be trained using the time-series data associated with the donors selected during the operation 1808 as well as time-series data associated with the target. As explained above, training the synthetic control model may include determining weights for calculating a weighted average of the donor time-series data that mirrors the target time-series data as close as possible during the first period before the intervention, such as by performing linear regression without intercept and allowing for negative coefficients. In an example, the synthetic control model may estimate the causal effect of adding the additional feature to the target module in the media playback application. In an embodiment, the synthetic control model may be trained by a synthetic control engine.

The operation 1812 is performed to present a graphical representation of the synthetic control model. In an example, the graphical representation is based on an observed outcome and the synthetic control model. As described above, FIGS. 8-17 illustrate examples of graphical representations that may be displayed. In an embodiment, a synthetic control server may cause a computing device to present the graphical representation.

In alternative embodiments, additional operations may be performed during the method 1800. For example, one or more bias bounds may be calculated for the synthetic control model. In an embodiment, the graphical representation is further based on the one or more bias bounds, as described above.

The various embodiments described above are provided by way of illustration only and should not be construed to limit the claims attached hereto. Those skilled in the art will readily recognize various modifications and changes that may be made without following the example embodiments and applications illustrated and described herein, and without departing from the full scope of the following claims.

What is claimed is:

1. A method for selecting training donors for a synthetic control model, the method comprising:
determining a set of potential donors, each potential donor associated with timeseries data comprising data before an intervention and data after the intervention;
for each potential donor in the set of potential donors:
determining an expected post-intervention value for the potential donor; and
comparing the expected post-intervention value to an actual post-intervention value for the potential donor;
selecting a set of training donors from the set of potential donors based on the comparisons;
training a synthetic control model on the timeseries data associated with each training donor in the set of training donors; and
causing a visual output device of a computing device to present a graphical representation based, at least in part, on an observed outcome and the synthetic control model.

2. The method of claim 1, wherein the expected post-intervention value for the potential donor is calculated based on the timeseries data for one or more other potential donors in the set of potential donors and one or more error distributions.

3. The method of claim 2, wherein the one or more error distributions include at least one of error distributions for the one or more other potential donors or error distributions for one or more latent variables.

4. The method of claim 1, wherein the expected post-intervention value for the potential donor is an expected value at a time of the intervention.

5. The method of claim 1, further comprising:
computing a bias for a synthetic control unit from the synthetic control model,
wherein the graphical representation is further based on the bias.

6. The method of claim 5, wherein computing the bias for the synthetic control unit from the synthetic control model includes:
selecting a weight from the synthetic control model;
for each training donor in the set of training donors, computing a difference between an average of the timeseries data before the intervention and an average of the timeseries data after the intervention;
selecting a difference from the computed differences; and
computing the bias, wherein the bias is a product of a number of training donors, the selected weight, and the selected difference.

7. The method of claim 5, wherein computing the bias for the synthetic control unit from the synthetic control model includes:
selecting a weight from the synthetic control model;
determining a set of excluded donors, the set of excluded donors including one or more potential donors that are not included in the set of training donors;
for each excluded donor in the set of excluded donors, computing a difference between an average of the timeseries data before the intervention and an average of the timeseries data after the intervention;
selecting a difference from the computed differences; and
computing the bias, wherein the bias is a product of a number of training donors, the selected weight, and the selected difference.

8. The method of claim 5, wherein computing the bias for the synthetic control unit from the synthetic control model includes:
selecting a weight from the synthetic control model;
for each training donor in the set of training donors, estimating a spillover value based on the intervention;
selecting a spillover value from the estimated spillover values; and
computing the bias, wherein the bias is a product of a number of training donors, the selected weight, and the selected spillover value.

9. The method of claim 1, wherein selecting the set of training donors from the set of potential donors based on the comparison includes:
ranking the potential donors in the set of potential donors based on the comparisons; and
selecting a predetermined number of training donors from the set of potential donors based on the ranking.

10. The method of claim 9, wherein the potential donors are ranked based on differences between the expected post-intervention values and the actual post-intervention values, wherein a higher rank correlates with a smaller difference between the expected post-intervention values and the actual post-intervention values.

11. The method of claim 1, wherein the set of training donors includes one or more potential donors for which a difference between the expected post-intervention value and the actual post-intervention value for the one or more potential donors at the time of the intervention is less than a predetermined threshold.

12. A system for selecting training donors for a synthetic control model, the system comprising:
one or more processors; and
one or more computer-readable storage devices storing data instructions that, when executed by the one or more processors, cause the system to:
determine a set of potential donors, each potential donor associated with timeseries data comprising data before an intervention and data after the intervention;
for each potential donor in the set of potential donors:
determine an expected post-intervention value for the potential donor; and
compare the expected post-intervention value to an actual post-intervention value for the potential donor;
select a set of training donors from the set of potential donors based on the comparisons;
train a synthetic control model on the timeseries data associated with each training donor in the set of training donors; and
cause a visual output device of a computing device to present a graphical representation based, at least in part, on an observed outcome and the synthetic control model.

13. The system of claim 12, wherein the graphical representation includes a difference between the observed outcome and a synthetic control unit of the synthetic control model.

14. The system of claim 12, wherein the graphical representation includes a cumulative difference between the observed outcome and a synthetic control unit of the synthetic control model.

15. The system of claim 12, wherein the graphical representation includes one or more of a table or a line chart.

16. A non-transitory computer-readable medium having stored thereon data instructions that, when executed by one or more processors, cause the one or more processors to:
determine a set of potential donors, each potential donor associated with timeseries data comprising data before an intervention and data after the intervention;
for each potential donor in the set of potential donors:
determine an expected post-intervention value for the potential donor; and
compare the expected post-intervention value to an actual post-intervention value for the potential donor;
select a set of training donors from the set of potential donors based on the comparisons;
train a synthetic control model on the timeseries data associated with each training donor in the set of training donors; and
cause a visual output device of a computing device to present a graphical representation based, at least in part, on an observed outcome and the synthetic control model.

17. The computer-readable medium of claim 16, further storing instructions that, when executed by the one or more processors, cause the one or more processors to:
select a weight from the synthetic control model;
determine a set of excluded donors, the set of excluded donors including one or more potential donors that are not included in the set of training donors;
for each excluded donor in the set of excluded donors, compute a difference between an average of the timeseries data before the intervention and an average of the timeseries data after the intervention;
select a difference from the computed differences; and
compute a bias, wherein the bias is a product of a number of training donors, the selected weight, and the selected difference,
wherein the graphical representation is further based on the bias.

18. The computer-readable medium of claim 17, wherein the selected weight has a maximum absolute value from among weights in the synthetic control model, and
wherein the selected difference has a maximum absolute value from among the computed differences.

19. The computer-readable medium of claim 16, further storing instructions that, when executed by the one or more processors, cause the one or more processors to:
select a weight from the synthetic control model;
for each training donor in the set of training donors, estimate a spillover value based on the intervention;
select a spillover value from the estimated spillover values; and
compute a bias, wherein the bias is a product of a number of training donors, the selected weight, and the selected spillover value,
wherein the graphical representation is further based on the bias.

20. The computer-readable medium of claim 19, wherein the selected weight has a maximum absolute value from among weights in the synthetic control model, and
wherein the selected spillover value has a maximum absolute value from among the estimated spillover values.
